(19) United States
(12) Plant Patent Application Publication
(10) Pub. No.: US 2016/0360678 P1
(43) Pub. Date: Dec. 8, 2016

(54) Pelargonium plant named 'KLEPZ15357'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/998,909

(22) Filed: Mar. 2, 2016

(30) Foreign Application Priority Data
Jun. 3, 2015 (CH) 15-3012

(51) Int. Cl. A01H 5/00

(52) U.S. Cl. PLT/330

(57) ABSTRACT

A new pelargonium plant particularly distinguished by red flowers, and an upright and compact growth habit, is disclosed.

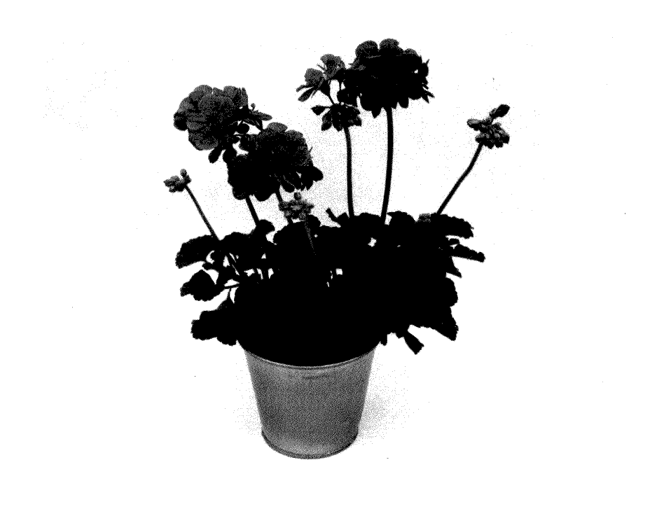

### CROSS-REFERENCE TO RELATED APPLICATIONS

This application claims priority to and the benefit of Switzerland Plant Breeders Rights application filed on Jun. 3, 2015, Application No. 15-3012, the entire contents of which are incorporated herein by reference for all purposes.

An application with the European Community Plant Variety Office was filed on July 2, 2015, File No. 2015-1525.

### GENUS AND SPECIES

Pelargonium zonale

### VARIETY DENOMINATION

‘KLEPZ15357’

### BACKGROUND OF THE NEW PLANT

The present invention comprises a new and distinct variety of pelargonium, botanically known as Pelargonium zonale and hereinafter referred to by the variety name ‘KLEPZ15357’. ‘KLEPZ15357’ originated from a cross-combination in June 2009 in Stuttgart, Germany between the female pelargonium variety ‘PZ-2002-0037’ (unpatented) and the proprietary male pelargonium variety ‘PZ-2005-0031’ (unpatented).

Seeds from the cross-combination in June 2009 in Stuttgart, Germany were obtained. The seeds were sown and plants were grown for evaluation. A single plant was selected in May 2010 and subsequently named ‘KLEPZ15357’. In August 2010 ‘KLEPZ15357’ was first vegetatively propagated by vegetative cuttings from stems. ‘KLEPZ15357’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.

1. Red flowers; and

2. An upright and compact growth habit.

### DESCRIPTION OF THE PHOTOGRAPH

This new pelargonium plant is illustrated by the accompanying photograph which shows an overall view of the plant. The photograph is of an approximately ten-week-old plant grown in Stuttgart, Germany under greenhouse conditions in May 2014. The colors shown are as true as can be reasonably obtained by conventional photographic procedures.

### DESCRIPTION OF THE NEW VARIETY

The following detailed descriptions set forth the distinctive characteristics of ‘KLEPZ15357’. The data which define these characteristics were collected from asexual reproductions carried out in Stuttgart, Germany. Data was collected on plants grown 2-months after planting in Stuttgart, Germany under greenhouse conditions in June 2015. Color references are to The R.H.S. Colour Chart of The Royal Horticultural Society of London (R.H.S.), edition (2007).
Classification:
   Family.—Geraniaceae.
   Botanical.—Pelargonium zonale.
   Common.—Pelargonium.
   Designation.—‘KLEPZ15357’.
Parentage:
   Female parent.—The female pelargonium variety ‘PZ-2002-0037’ (unpatented).
   Male parent.—The proprietary male pelargonium variety ‘PZ-2005-0031’ (unpatented).
Plant:
   Form.—Bushy and round.
   Habit.—Upright and compact.
   Height.—13.0 cm to 15.0 cm.
   Spread.—17.0 cm to 20.0 cm.
   Natural flowering season.—Spring to autumn.
   Propagation type.—Vegetative cuttings.
   Time to produce a finished flowering plant.—10 weeks.
   Time to initiate and develop roots.—17 days.
   Root description.—White, RHS 155C, and fine.
Stems:
   Quantity of stems per plant.—4.
   Color.—RHS 137A.
   Surface texture.—Sparsely pubescent.
   Stem form.—Round.
   Diameter.—0.9 cm.
   Length.—8.0 cm to 10.0 cm.
   Internode length.—1.0 cm to 2.0 cm.
   Anthocyanin.—Present, color is RHS 174A.
Leaves:
   Arrangement.—Alternate.
   Shape.—Kidney-shaped.
   Apex.—Obtuse, round.
   Base.—Open.
   Margin.—Crenate.
   Surface texture.—Upper surface: Moderately pubescent Lower surface: Moderately pubescent.
   Length.—4.0 cm to 5.0 cm.
   Width.—5.0 cm to 6.0 cm.
   Color.—Upper surface: RHS N137B Lower surface: RHS 137A.
   Petiole.—Length: 5.0 cm to 6.0 cm Diameter: 0.4 cm Color: RHS 137A Strength: Medium.
   Venation pattern.—Palmate.
   Venation color.—Upper surface: RHS 137B Lower surface: RHS 137D.
Inflorescence:
   Lastingness of flowers on the plant.—6 days.
   Umbels.—Quantity of umbels per plant: 5 Length: 4.0 cm Diameter: 7.0 cm.
   Individual florets of the umbel.—Quantity of florets per umbel: 7 to 9 Depth: 2.0 cm Diameter: 4.0 cm.
   Fragrance.—Absent.
Flower buds:
   Cluster shape.—Round.
   Cluster size.—Length: 3.0 cm Width: 3.0 cm.
   Surface texture.—Rough, sparsely pubescent.
   Bud.—Height: 1.0 cm to 1.5 cm Width: 0.5 cm Shape: Elliptic Apex: Peaked Base: Blunt Margin: Entire Color: RHS 137A.
Petals:
   Quantity per flower.—6 to 7 and petaloids.
   Shape.—Drop-shaped.
   Apex.—Round.
   Base.—Attenuate.
   Margin.—Entire.
   Length.—2.0 cm.
   Width.—3.0 cm.
   Surface texture.—Upper surface: Smooth Lower surface: Smooth.
   Color of lobes.—Upper lobes: Upper surface: RHS N30A Lower surface: RHS 33A. Lower lobes: Upper surface: RHS N30A Lower surface: RHS 33A.
Petaloids:
   Quantity per flower.—2 to 3.
   Shape.—Moderate.
   Apex.—Round.
   Base.—Attenuate.
   Margin.—Obtuse, blunt.
   Texture (upper and lower surfaces).—Smooth.
   Color.—Upper surface: RHS N30A Lower surface: RHS 33A.
Sepals:
   Number per flower.—5.
   Shape.—Elliptic.
   Apex.—Peaked.
   Base.—Obtuse.
   Margin.—Entire.
   Surface texture.—Upper surface: Smooth Lower surface: Moderately pubescent.
   Color.—Upper surface: RHS 137C and RHS 176C Lower surface: RHS 137A and RHS 176C.
   Length.—0.8 cm.
   Width.—0.3 cm.
Pedicel:
   Length.—1.0 cm to 1.6 cm.
   Diameter.—0.1 cm.
   Color.—RHS 176B.
   Surface texture.—Sparsely pubescent.
   Strength.—Medium.
Peduncle:
   Length.—8.0 cm to 10.0 cm.
   Diameter.—0.4 cm.
   Color.—RHS 176B and RHS 137A.
   Surface texture.—Sparsely pubescent.
   Strength.—Medium.
Reproductive organs:
   Stamens.—Quantity of stamens per flower: 1 to maximum of 5 Anther length: 0.15 cm Filament length: 0.8 cm to 1.0 cm Filament color: RHS 49D Pollen amount: Moderate Pollen color: RHS 28C.
   Pistils.—Number: 1 Stigma color: RHS 63A Style length: 0.6 cm Style color: RHS 63A Ovary arrangement: Central Ovary color: RHS 145C Ovary diameter: 0.2 cm Ovary length: 0.4 cm
Fruit and seed set: None observed
Disease and pest/insect resistance: None observed

### COMPARISON WITH PARENTAL LINES AND KNOWN VARIETY

‘KLEPZ15357’ is distinguished from its parents as described in Table 1:

TABLE 1
Comparison with Parental Lines

| | | Female parent | Male Parent |
|---|---|---|---|
| Characteristic | ‘KLEPZ15357’ | ‘PZ-2002-0037’ | ‘PZ-2005-0031’ |
| Flower color | Red | Orange | Dark-red |
| Growth | Compact | Compact | Strong |
| Production | Good | Medium | Medium |

‘KLEPZ15357’ can be compared to the commercial variety ‘KLEP04130’ (U.S. Plant Pat. No. 18,023), also commercially known as MOONLIGHT ‘Paprika’. Differences between the two varieties are described in Table 2:

TABLE 2
Comparison with Similar Variety

| | | Commercial line |
|---|---|---|
| Characteristic | ‘KLEPZ15357’ | ‘KLEP04130’ |
| Growth | Compact | Strong |
| Color | Red | Orange-red |

We claim:

1. A new and distinct variety of pelargonium plant named ‘KLEPZ15357’ as illustrated and described herein.
